(19) United States
(12) Patent Application Publication
(10) Pub. No.: US 2026/0130905 A1
(43) Pub. Date: May 14, 2026

(54) TOPICAL FORMULATIONS OF DEUCRAVACITINIB

(71) Applicant: Bristol-Myers Squibb Company, Princeton, NJ (US)

(72) Inventors: Monika Lavan, Princeton, NJ (US); Umesh Kestur, New Brunswick, NJ (US); Divyakant S. Desai, Princeton, NJ (US); Charles Evans, Godalming (GB); James Sayer, Aldershot (GB); Florencia Guidali, London (GB)

(21) Appl. No.: 18/705,178

(22) PCT Filed: Oct. 27, 2022
(86) PCT No.: PCT/US2022/048088

§ 371 Date: Apr. 26, 2024

Related U.S. Application Data
(60) Provisional application No. 63/273,111, filed on Oct. 28, 2021.

(51) Int. Cl. A61K 31/501; A61K 9/00; A61K 9/06; A61K 47/10; A61K 47/22; A61K 47/32

(52) U.S. Cl. CPC A61K 31/501; A61K 9/0014; A61K 9/06; A61K 47/10; A61K 47/22; A61K 47/32

(57) ABSTRACT

Described herein are topical formulations of deucravacitinib and methods of making such formulations. Also described are methods of treatment involving the topical administration of such formulations.

### FIELD OF THE INVENTION

The present invention generally relates to topical formulations of deucravacitinib, which is a tyrosine kinase 2 (TYK2) inhibitor. Such formulations are useful in the treatment and management of diseases such as, for example, psoriasis, psoriatic arthritis, lupus, and alopecia areata.

### BACKGROUND

Deucravacitinib is a selective TYK2 inhibitor that has shown efficacy in the treatment of certain inflammatory and autoimmune diseases such as psoriasis. While deucravacitinib has been formulated for oral administration, there is a need for topical dosage forms of deucravacitinib. The present invention addresses such need.

### SUMMARY OF THE INVENTION

Described herein are novel topical pharmaceutical compositions comprising deucravacitinib. Deucravacitinib is also known as 6-(cyclopropanecarboxamido)-4-((2-methoxy-3-(1-methyl-1H-1,2,4-triazol-3-yl)phenyl)amino)-N-(methyl-d₃)pyridazine-3-carboxamide, a compound having the structure of Formula (I):

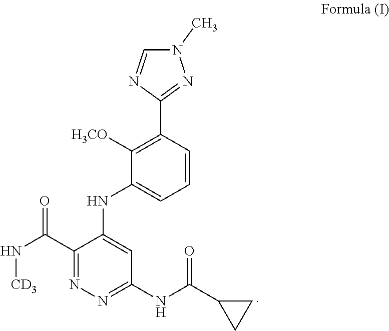 [SMILES: [2H]C([2H])([2H])NC(=O)c1nnc(NC(=O)C2CC2)cc1Nc1cccc(-c2ncn(C)n2)c1OC]

Deucravacitinib is a selective TYK2 inhibitor useful for the treatment of certain inflammatory and autoimmune diseases such as psoriasis, psoriatic arthritis, lupus, lupus nephritis, Sjögren's syndrome, ulcerative colitis, Crohn's disease, ankylosing spondylitis, and alopecia areata. For certain such diseases, it may be desirable to administer one or more therapeutic agents topically during treatment. Developing topical formulations of deucravacitinib, however, has been challenging due to, for example, deucravacitinib's poor solubility in water.

The present disclosure provides formulations of deucravacitinib that are suitable for topical administration, and in which deucravacitinib completely dissolves and is stable upon storage. By formulating deucravacitinib as described herein, deucravacitinib can be supplied in topical dosage forms (e.g., creams, ointments, gels) that can be used in the treatment of inflammatory and autoimmune diseases in which topical administration of deucravacitinib may be desirable (diseases such as, e.g., psoriasis, psoriatic arthritis, systemic lupus erythematosus, and alopecia areata).

In certain embodiments, a topical formulation of deucravacitinib comprises, in addition to deucravacitinib, an ether solvent. The ether solvent may be an ether solvent selected from polyethylene glycol 400 (PEG 400), diethylene glycol monoethyl ether (DEGEE), dimethyl isosorbide (DMI), d-α-tocopheryl polyethylene glycol 1000 succinate (TPGS), and propylene glycol; in certain embodiments, the ether solvent is PEG 400. In further embodiments, the topical formulation of deucravacitinib comprises deucravacitinib and two ether solvents, and in certain embodiments, the two ether solvents are selected from polyethylene glycol 400 (PEG 400), diethylene glycol monoethyl ether (DEGEE), dimethyl isosorbide (DMI), d-α-tocopheryl polyethylene glycol 1000 succinate (TPGS), and propylene glycol. In still further embodiments, the topical formulation comprises deucravacitinib and three ether solvents, wherein the three ether solvents are selected from polyethylene glycol 400 (PEG 400), diethylene glycol monoethyl ether (DEGEE), dimethyl isosorbide (DMI), d-α-tocopheryl polyethylene glycol 1000 succinate (TPGS), and propylene glycol. In certain embodiments, the formulation comprises at least PEG 400. For example, in embodiments where there are two ether solvents included in the formulation, the ether solvents are PEG 400 and DEGEE, or are PEG 400 and DMI; in embodiments where there are three ether solvents included in the formulation, the ether solvents can be PEG 400, DEGEE, and DMI. In any of the aforementioned embodiments, the topical formulation of deucravacitinib may be an aqueous formulation (e.g., an aqueous gel or cream) and may further comprise an acidic buffer.

Embodiments of the invention also relate to a topical formulation of deucravacitinib comprising deucravacitinib, an ether solvent, and an acidic buffer. In some embodiments, the ether solvent is selected from polyethylene glycol 400 (PEG 400), diethylene glycol monoethyl ether (DEGEE), dimethyl isosorbide (DMI), d-α-tocopheryl polyethylene glycol 1000 succinate (TPGS), and propylene glycol; in certain embodiments, the ether solvent is PEG 400. Further embodiments of the invention relate to a topical formulation comprising deucravacitinib, two ether solvents, and an acidic buffer. In certain such embodiments, the two ether solvents are selected from polyethylene glycol 400 (PEG 400), diethylene glycol monoethyl ether (DEGEE), dimethyl isosorbide (DMI), d-α-tocopheryl polyethylene glycol 1000 succinate (TPGS), and propylene glycol and may be, for example, PEG 400 and DEGEE, or PEG 400 and DMI. In further embodiments, the topical formulation comprises deucravacitinib, three ether solvents, and an acidic buffer.

In any of the aforementioned embodiments, one or more additional excipients (e.g., thickening agents, preservatives, etc.) as described herein may be included in the formulation. In addition, in any of the aforementioned embodiments, the deucravacitinib may be present in the formulation in an amount ranging from about 0.075% w/w to about 1.1% w/w, based on total weight of the composition. In certain such embodiments, the deucravacitinib is present in the formulation in an amount ranging from about 0.3% w/w to about 1.1% w/w, based on total weight of the composition.

Methods of preparing topical dosage forms of deucravacitinib are also described herein.

### DETAILED DESCRIPTION

The present disclosure relates to topical pharmaceutical compositions comprising deucravacitinib. The terms “topical pharmaceutical composition,” “topical composition,” “topical formulation,” and “topical dosage form” as used herein generally refer to a composition that is pharmaceutically acceptable and that is suitable for administering deucravacitinib topically to a subject. Such compositions include but are not limited to creams, ointments, gels, foams, sprays, lotions, solutions, emulsions, suspensions, mists, aerosols, unguents, and pastes. The topical compositions generally comprise, in addition to deucravacitinib, at least one ether solvent. The compositions may comprise one or more additional excipients as described herein. Such topical pharmaceutical compositions comprising deucravacitinib may be used to treat diseases such as psoriasis, psoriatic arthritis, systemic lupus erythematosus, and alopecia areata, for example.

Deucravacitinib and methods of making deucravacitinib are disclosed in U.S. Pat. No. RE47,929 E, the contents of which are hereby incorporated by reference herein in their entirety. Deucravacitinib is also known as 6-(cyclopropanecarboxamido)-4-((2-methoxy-3-(1-methyl-1H-1,2,4-triazol-3-yl)phenyl)amino)-N-(methyl-d₃)pyridazine-3-carboxamide, a compound having the structure of Formula (I):

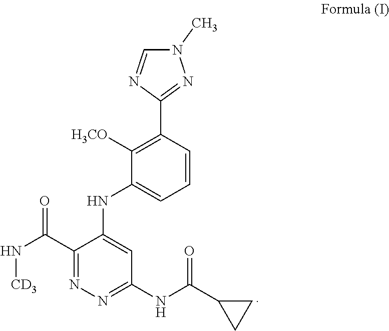 [SMILES: [2H]C([2H])([2H])NC(=O)c1nnc(NC(=O)C2CC2)cc1Nc1cccc(-c2ncn(C)n2)c1OC]

The deucravacitinib used to make any of the formulations described herein may comprise deucravacitinib in amorphous form and/or in crystalline form. Crystalline forms of deucravacitinib (and of salts of deucravacitinib) are described in, for example, International Application Nos. PCT/US2018/025114, PCT/US2019/034534, and PCT/US2020/036727 (published as WO 2018/183656, WO 2019/232138, and WO 2020/251911, respectively), the entire contents of each of which are hereby incorporated by reference herein.

Deucravacitinib is a selective TYK2 inhibitor currently in clinical trials for the treatment of inflammatory and autoimmune diseases such as psoriasis, psoriatic arthritis, lupus, lupus nephritis, Sjögren's syndrome, ulcerative colitis, Crohn's disease, and ankylosing spondylitis. TYK2 is a member of the Janus kinase (JAK) family of nonreceptor tyrosine kinases and has been shown to be critical in regulating the signal transduction cascade downstream of receptors for IL-12, IL-23, and type I interferons in both mice (Ishizaki, M. et al., “Involvement of tyrosine kinase-2 in both the IL-12/Th1 and IL-23/Th17 axes in vivo,” J. Immunol., 187:181-189 (2011); Prchal-Murphy, M. et al., “TYK2 kinase activity is required for functional type I interferon responses in vivo,” PLOS One, 7: e39141 (2012)) and humans (Minegishi, Y. et al., “Human tyrosine kinase 2 deficiency reveals its requisite roles in multiple cytokine signals involved in innate and acquired immunity,” Immunity, 25:745-755 (2006)). TYK2 mediates the receptor-induced phosphorylation of members of the STAT family of transcription factors, an essential signal that leads to the dimerization of STAT proteins and the transcription of STAT-dependent pro-inflammatory genes. TYK2-deficient mice are resistant to experimental models of colitis, psoriasis, and multiple sclerosis, demonstrating the importance of TYK2-mediated signaling in autoimmunity and related disorders (Ishizaki, M. et al., “Involvement of tyrosine kinase-2 in both the IL-12/Th1 and IL-23/Th17 axes in vivo,” J. Immunol., 187:181-189 (2011); Oyamada, A. et al., “Tyrosine kinase 2 plays critical roles in the pathogenic CD4 T cell responses for the development of experimental autoimmune encephalomyelitis,” J. Immunol., 183:7539-7546 (2009)).

In humans, individuals expressing an inactive variant of TYK2 are protected from multiple sclerosis and possibly other autoimmune disorders (Couturier, N. et al., “Tyrosine kinase 2 variant influences T lymphocyte polarization and multiple sclerosis susceptibility,” Brain, 134:693-703 (2011)). Genome-wide association studies have shown other variants of TYK2 to be associated with autoimmune disorders such as Crohn's disease, psoriasis, systemic lupus erythematosus, and rheumatoid arthritis, further demonstrating the importance of TYK2 in autoimmunity (Ellinghaus, D. et al., “Combined Analysis of Genome-wide Association Studies for Crohn Disease and Psoriasis Identifies Seven Shared Susceptibility Loci,” Am. J. Hum. Genet., 90:636-647 (2012); Graham, D. et al., “Association of polymorphisms across the tyrosine kinase gene, TYK2 in UK SLE families,” Rheumatology (Oxford), 46:927-930 (2007); Eyre, S. et al., “High-density genetic mapping identifies new susceptibility loci for rheumatoid arthritis,” Nat. Genet., 44:1336-1340 (2012)).

Deucravacitinib is poorly soluble in water, with a solubility of about 0.009 mg/mL or 0.00009% w/w. Water, however, is an essential component in many topical formulations, such as aqueous gel and cream formulations. Described herein are various topical formulations comprising deucravacitinib in which the deucravacitinib is completely dissolved, notwithstanding its poor solubility in water. Moreover, the deucravacitinib in such topical formulations is stable upon storage.

In some embodiments, a topical formulation of deucravacitinib comprises deucravacitinib and at least one solvent belonging to the ether class. Such solvents include, for example, polyethylene glycol (PEG) (such as, e.g., PEG 200, PEG 300, PEG 400, and PEG 600), diethylene glycol monoethyl ether (DEGEE), dimethyl isosorbide (DMI), d-α-tocopheryl polyethylene glycol 1000 succinate (TPGS), and propylene glycol. In certain embodiments, the formulation comprises at least two ether solvents. In further embodiments, the solvents included in the formulation comprise PEG (e.g., PEG 400) and at least one of DEGEE, DMI, TPGS, and propylene glycol. The formulation may further comprise an acidic buffer and/or water.

In certain embodiments, a topical formulation comprises deucravacitinib, PEG 400, and at least one of DEGEE and DMI. For example, the formulation may comprise deucravacitinib, PEG 400, and DEGEE (and not also DMI), or the formulation may comprise deucravacitinib, PEG 400, and DMI (and not also DEGEE). In some embodiments, a topical formulation comprises deucravacitinib, PEG 400, DMI, and DEGEE.

In some embodiments, a topical formulation of deucravacitinib comprises an aqueous component (e.g., the topical formulation may be an aqueous gel or cream formulation). In certain such embodiments, the topical formulation comprises deucravacitinib, an ether solvent, and an acidic buffer. As demonstrated herein, using an ether-based solvent (or two or more ether-based solvents), and/or adjusting the pH with an acidic buffer, provides for increased solubility of deucravacitinib. The ether solvent may be PEG (e.g., PEG 400), DEGEE, DMI, TPGS, or propylene glycol. In certain embodiments, the ether solvent is PEG 400. In further embodiments, the topical formulation comprises deucravacitinib, at least two ether solvents, and an acidic buffer. One of such ether solvents may be PEG (e.g., PEG 400), and the other ether solvent may be DEGEE, DMI, TPGS, or propylene glycol. For example, a topical formulation may comprise deucravacitinib, PEG 400, DEGEE, and an acidic buffer, or it may comprise deucravacitinib, PEG 400, DMI, and an acidic buffer. In other embodiments, the topical formulation comprises deucravacitinib, PEG 400, DEGEE, DMI, and an acidic buffer. Suitable acidic buffers are known in the art and include, for example, hydrochloric acid solution, citric acid, phosphoric acid, and other acidifying agents.

The total amount of ether solvent(s) in a topical composition of deucravacitinib as described herein may range from about 30% w/w to about 98% w/w, based on total weight of the composition. For example, in non-aqueous formulations (such as non-aqueous gels and ointments), the one or more ether solvents may make up about 60% w/w to about 98% w/w of the total % w/w of the composition. In certain embodiments, the amount of the one or more ether solvents in a non-aqueous formulation ranges from about 70% w/w to about 98% w/w, based on total weight of the composition. In aqueous formulations (such as aqueous gels, emulsified gels, and creams), the one or more ether solvents may account for about 40% w/w to about 90% w/w, while the aqueous component (e.g., water and/or buffer solution) may account for about 5% w/w to about 30% w/w, of the total % w/w of the composition. In certain embodiments, for example, the amount of the one or more ether solvents may range from about 50% w/w to about 55% w/w, and the amount of the aqueous component (e.g., water and/or buffer) may range from about 10% w/w to about 25% w/w, based on total weight of the composition (while other excipients make up the remaining % w/w). In other examples, the one or more ether solvents account for about 85% w/w to about 90% w/w, and the aqueous component (e.g., water or acidic buffer) accounts for about 5% w/w to about 10% w/w (e.g., about 7% w/w), of the total % w/w of the composition.

Topical formulations comprising deucravacitinib and one or more ether solvents may comprise one or more additional pharmaceutically acceptable excipients. Such excipients include, for example, thickening agents (e.g., carbomer or a carbomer derivative, cellulose and derivatives thereof, anionic polymers); emulsifiers and surfactants; humectants; viscosity enhancers; chelating agents, antioxidants and other preservatives; foaming agents; ointment base components; and propellants.

In certain embodiments, a topical formulation comprising deucravacitinib and at least one ether solvent as described above (e.g., a topical formulation comprising deucravacitinib and two or three ether solvents) further includes one or more of the following excipients:
excipients providing oil phase: e.g., castor oil, medium chain triglyceride, mono/diglycerides, petrolatum, beeswax
emulsifiers and surfactants: e.g., polysorbate 80, emulsifying wax, Brij 20, glycerol monostearate, cationic and anionic surfactants
solvents and solubilizers: e.g., cyclodextrins
thickening agents: e.g., cetostearyl alcohol, PEG 1500, cellulose and derivatives thereof, carbomer and derivatives thereof (for example, carbomer 910, 940, 941, 1342, 934P, and 974P)
preservatives: e.g., phenoxyethanol, benzyl alcohol
antioxidants: e.g., butylated hydroxyanisole (BHA), butylhydroxytoluene (BHT), and similar excipients (for example, tert-butylhydroquinone (TBHQ))
complexation agents: e.g., ethylenediaminetetraacetic acid (EDTA)
permeation enhancers: e.g., modified lipid/fatty acid solubilizers, salts of fatty acids
Table 3 below includes additional excipients that are suitable for deucravacitinib topical formulations as described herein. The function ascribed to a particular excipient is not intended to be limiting, such that an excipient may serve several functions or provide several benefits.

The topical formulations described herein can comprise varying amounts of deucravacitinib. For example, the amount of deucravacitinib included in any of the embodiments described herein can be from about 0.075% w/w to about 1.1% w/w, based on total weight of the composition (e.g., in certain embodiments, the deucravacitinib is present in the formulation in an amount that is about 0.075% w/w, or in an amount that is about 1.1% w/w, or in any amount in between (such as 0.3% w/w), based on total weight of the composition). Generally, it is desirable that the deucravacitinib be completely dissolved in the formulation; such dissolution of deucravacitinib can be determined by, e.g., microscopically examining a sample of the formulation. To avoid precipitation of deucravacitinib in a formulation, the upper limit of drug loading can be set at 80% of saturated solubility of deucravacitinib in the particular solvent(s) of the formulation. Because the different solvent combinations described herein provide a range of saturated solubilities for deucravacitinib, the amount of deucravacitinib generally will not be limited by formulation constraints and instead can be based on the therapeutic window and the minimum effective dose required for treatment. To provide for complete dissolution of higher concentrations of deucravacitinib, a combination of ether solvents, and/or the addition of an acidic buffer (such as hydrochloric acid, citric acid, etc.), can be used during formulation to provide for higher saturated solubilities of deucravacitinib.

The present invention also relates to methods of administering a topical composition comprising deucravacitinib as described herein. For example, aspects of the invention relate to methods of treating psoriasis, psoriatic arthritis, lupus, or alopecia areata in a subject comprising administering to the subject a topical composition comprising deucravacitinib as described herein. Topical administration of deucravacitinib may provide several advantages over oral administration such as dose reduction, fewer and/or less severe side-effects, and improved efficacy (e.g., because of local delivery of a therapeutic amount of deucravacitinib), and improved patient compliance.

Some embodiments of the invention relate to a method of treating or preventing psoriasis in a subject, the method comprising topically administering to a skin area of the subject a topical composition comprising deucravacitinib as described herein. Generally, the skin area is, or is prone to be, affected by psoriasis (for example, the skin area exhibits, or is prone to exhibit, a symptom associated with psoriasis such as, e.g., psoriatic plaques).

In addition, embodiments of the invention relate to a method of treating psoriatic arthritis in a subject, or of preventing a symptom associated with psoriatic arthritis in a subject, the method comprising administering to a skin area of the subject a topical composition comprising deucravacitinib as described herein.

Other embodiments of the present invention relate to a method of treating alopecia areata in a subject, or of preventing hair loss associated with alopecia areata in a subject, the method comprising administering to a skin area of the subject a topical composition comprising deucravacitinib as described herein. For a subject suffering from alopecia areata, topical administration of the composition to affected sites of the skin may promote hair regrowth in such sites. For methods of preventing a relapse of hair loss associated with alopecia areata, a topical pharmaceutical composition comprising deucravacitinib as described herein may be administered to a subject who has previously experienced hair loss associated with alopecia areata, wherein the composition is administered to, for example, certain areas of the scalp that may be prone to hair loss.

Further embodiments of the invention relate to a method of treating systemic lupus erythematosus in a subject, the method comprising administering to a skin area of the subject a topical composition comprising deucravacitinib as described herein.

In some embodiments, a topical composition comprising deucravacitinib as described herein is administered to a subject (e.g., a subject suffering from psoriasis, a subject suffering from psoriatic arthritis, etc.) over the course of three days, seven days, ten days, fourteen days, or longer. In certain embodiments, administration may continue for weeks or months (e.g., one month, three months, etc.). In addition, administration of a topical composition comprising deucravacitinib as described herein may comprise administering the topical composition once daily, twice daily, or thrice daily, on consecutive days; alternatively, administration may be every other day (e.g., on days 1 and 3, but not on day 2), every other two days (e.g., on days 1 and 4, but not on days 2 and 3), etc.

In the context of the present invention, subjects, and in particular human subjects, may also be referred to as patients.

Any definitions set forth herein take precedence over definitions set forth in any patent, patent application, and/or patent application publication incorporated herein by reference. The text of any patent, patent application, patent application publication, or other material that has been incorporated by reference is incorporated by reference only to the extent that no conflict exists between such text and the present specification; in the event of a conflict, any conflicting text is specifically not incorporated by reference.

### EXAMPLES

The invention will be further described by the following examples. The examples serve only to illustrate the invention and its practice. The examples are not to be construed as limitations on the scope or spirit of the invention.

All measurements are subject to experimental error, consistent with the spirit of the invention.

### Example 1—Solubility of Deucravacitinib

The solubility of deucravacitinib was examined in various solvents. PEG 400 provided solubility of 0.64% w/w. Two other ether solvents, DEGEE and DMI, provided solubility of 0.62% w/w and 0.51% w/w, respectively. Deucravacitinib exhibited high solubility in N-methyl-2-pyrrolidone (NMP) and dimethyl sulfoxide (DMSO). As these solvents can, in some instances, result in skin irritation, they were not included in the development of the formulations described later in the Examples.

TABLE 1
Solubility of deucravacitinib in different solvents

| | | Deucravacitinib |
|---|---|---|
| | | solubility |
| Solvent | Solvent class | (% w/w) |
| Polyethylene glycol (PEG) | Ether | 0.64 |
| 400 | | |
| Diethylene glycol | Ether | 0.62 |
| monoethyl ether (DEGEE) | | |
| Dimethyl isosorbide (DMI) | Ether | 0.51 |
| Propylene glycol | Ether | 0.16 |
| Ethanol | Alcohol | 0.07 |
| Isopropanol | Alcohol | 0.02 |
| Octyldodecanol | Alcohol | 0.01 |
| Isopropyl myristate | Ester of fatty acid | 0.00 |
| Diisopropyl adipate | Ester of fatty acid | 0.01 |
| Caprylic/capric triglyceride | Medium chain triglyceride | 0.01 |
| Polysorbate 80 | Surfactant | 0.31 |
| Dimethyl sulfoxide | Organic solvent | 9.49 |
| (DMSO) | | |
| N-Methyl-2-pyrrolidone | Organic solvent | 10.09 |
| (NMP) | | |

### Example 2—Solubility of Deucravacitinib in Different Solvent Systems

The solubility of deucravacitinib was examined in various solvent systems that included water, as water is an essential component in aqueous gels and creams. The example solvent systems in Table 2 were designed for cream formulations. The total percentage of the solvent system was up to 80% of the total % w/w of the composition, with the remaining up to 20% reserved for oil component(s). Water or acidic buffer (an aqueous solution) accounted for 25% w/w of the total composition.

A synergistic effect was observed in solvent systems (systems comprising two or more solvents) comprising PEG 400 and at least one of two other solvents, DEGEE and DMI. The combination of PEG 400, DEGEE, and DMI in a three-solvent system (S3) provided the greatest solubility of deucravacitinib, as the solubility of deucravacitinib in the three-solvent system (S3) comprising PEG 400, DEGEE, and DMI was greater than the solubility of deucravacitinib observed in two-solvent systems (S1 and S2). A further increase in solubility was also observed for each of the solvent systems tested when an acidic buffer was used in place of unbuffered distilled water (S4, S5, and S6).

TABLE 2
Solubility of deucravacitinib in solvent systems

| | Solvent system | | | | | |
|---|---|---|---|---|---|---|
| | S1 | S2 | S3 | S4 | S5 | S6 |
| | % w/w | | | | | |
| Water | 25 | 25 | 25 | — | — | — |
| Acidic buffer: | — | — | — | 25 | 25 | 25 |
| Hydrochloric acid | | | | | | |
| solution, 0.1N | | | | | | |
| PEG 400 | 38 | 38 | 23 | 38 | 38 | 23 |
| DEGEE | 15 | — | 15 | 15 | — | 15 |
| DMI | — | 15 | 15 | — | 15 | 15 |
| Benzyl alcohol | 2 | 2 | 2 | 2 | 2 | 2 |
| Deucravacitinib | 0.51 | 0.48 | 0.72 | 0.77 | 0.83 | 1.33 |
| solubility (% w/w) | | | | | | |

### Example 3—Topical Formulations of Deucravacitinib

All deucravacitinib formulations were prepared by fully dissolving deucravacitinib in the solvent components. The maximum drug load used for each formulation (in Examples 4, 5, and 6) was limited to 80% of the saturated solubility of deucravacitinib in the particular solvents used in the formulation. Pharmaceutically acceptable excipients (such as oil phase in cream dosage forms, as well as humectant and skin conditioners) were added as non-solvent components. Brij S2, Brij S721, and poloxamer were used as surfactants. Benzyl alcohol was used as a preservative in formulations containing aqueous content. Citric acid, hydrochloric acid, and sodium hydroxide were used as pH modifiers. Microscopic examination was performed to confirm there was no precipitation of deucravacitinib or of excipients in the finished products.

TABLE 3
Ingredients used in exemplary topical
formulations of deucravacitinib

| Ingredient | Function |
|---|---|
| Deucravacitinib | Active ingredient |
| PEG 400 | Solvent |
| DEGEE | Solvent |
| DMI | Solvent |
| Propylene glycol | Solvent |
| Vitamin E Polyethylene Glycol Succinate | Solvent |
| (TPGS) | |
| Water | Aqueous phase |
| Benzyl alcohol | Preservative |
| Hydroxypropyl cellulose (HPC) | Thickening agent |
| Carbomer | Thickening agent |
| Carbomer interpolymer type A | Thickening agent |
| Cetyl alcohol | Thickening agent |
| PEG 3350 | Thickening agent |
| Stearic acid | Emulsion stabilizer |
| Mineral oil | Oil phase |
| Glycerol | Humectant |
| Caprylic/capric triglyceride | Skin conditioner |
| Modified lanolin | Skin conditioner |
| Isopropyl myristate | Non-volatile solvent |
| Poloxamer 407 | Surfactant |
| Brij S2, Brij S721 | Surfactant |
| Citric acid | pH modifier |
| 0.1N hydrochloric acid | pH modifier |
| 0.2M sodium hydroxide | pH modifier |
| Ammonia solution, 2.5% | pH modifier |

### Example 4—Preparation of Aqueous Gel Formulations

Aqueous gel formulations (Formulations F1, F2, F3, F4, and F5) were prepared via the following method:
1. Solvents and preservative (which in these embodiments was benzyl alcohol) were added to a vessel and were stirred until homogenous.
2. Deucravacitinib was added and the mixture was stirred until the deucravacitinib completely dissolved (based on visual observation).
3. Water was added dropwise while stirring.
4. For formulations containing carbomer as a thickening agent (Formulations F1, F2, and F4), carbomer was added slowly while stirring to achieve and maintain a vortex. Stirring continued until the carbomer was evenly dispersed. Next, pH adjustments were performed if needed and the remaining water was added at an amount sufficient to make up the total target batch size. For formulations containing HPC as a thickening agent (Formulations F3 and F5), pH of the formulation was adjusted if needed and the remaining water was added. Then, HPC was added while stirring. Stirring continued until the HPC was evenly dispersed.
5. For all formulations F1, F2, F3, F4, and F5, the resulting mixture was stirred overnight.
Microscopic examination was performed on a sample from each finished product to ensure there was no precipitation of deucravacitinib or of the excipients.

TABLE 4
Topical formulations of deucravacitinib - aqueous gels

| | F1 | F2 | F3 | F4 | F5 |
|---|---|---|---|---|---|
| Formulation | % w/w | | | | |
| Deucravacitinib | 0.66 | 0.41 | 0.76 | 0.67 | 1.10 |
| Water | 7.00 | 7.00 | — | 7.50 | — |
| Hydrochloric acid, | — | — | 7.50 | — | 7.50 |
| 0.1N | | | | | |
| PEG 400 | 61.59 | 67.84 | 61.24 | 46.33 | 45.90 |
| DEGEE | 25.00 | — | 25.00 | 25.00 | 25.00 |
| DMI | — | — | — | 15.00 | 15.00 |
| Propylene glycol | — | 20 | — | — | — |
| Carbomer | 0.75 | 0.75 | — | 1.00 | — |
| HPC | — | — | 1.00 | — | 1.00 |
| Benzyl alcohol | 2.00 | 2.00 | 2.00 | 2.00 | 2.00 |
| Sodium | adjust to | adjust to | | | |
| hydroxide, 0.2M | pH 6.0-6.5 | pH 6.0-6.5 | | | |
| Ammonia | — | — | adjust | — | adjust |
| solution, 2.5% v/v | | | to pH | | to pH |
| | | | 4.5-5.5 | | 4.5-5.5 |
| Additional water | q.s. | q.s. | q.s. | q.s. | q.s. |

q.s.: quantity sufficient

### Example 5—Preparation of Non-Aqueous Gel and Ointment Formulations

Non-aqueous gel formulations (Formulations F7, F8, and F9) were prepared via the following method:
1. PEG 400, DEGEE, and propylene glycol were added to a vessel according to target amounts in the formulations and were stirred until homogenous. For Formulation F8, 0.5 M citric acid was added to DEGEE before it was incorporated with other solvents.
2. For Formulation F8, TPGS was added and the resulting mixture was stirred at 65° C. until the TPGS melted.
3. Deucravacitinib was added and the mixture was stirred until the deucravacitinib completely dissolved (based on visual observation).
4. A thickening agent (which in these embodiments was hydroxypropyl cellulose (HPC)) was added slowly while stirring to achieve and maintain a vortex.
5. Stirring continued overnight.

An ointment (Formulation F6) was prepared as follows:
1. PEG 400 and DEGEE were added to a vessel and were stirred until homogenous.
2. PEG 3350 was added to a separate vessel and was heated to 70° C. until the PEG 3350 melted.
3. Deucravacitinib was added to the first vessel and was stirred until completely dissolved (based on visual observation). Then, the contents of the first vessel were heated to 70° C. for 5 minutes.
4. The melted PEG 3350 was added to the first vessel while stirring.
5. The resulting mixture in the first vessel was stirred manually with a spatula until cool.

TABLE 5
Topical formulations of deucravacitinib - non-
aqueous gels (F7, F8 and F9) and ointment (F6)

| | | | |
|---|---|---|---|
| F6 | F7 | F8 | F9 |

| | |
|---|---|
| Formulation | % w/w |

| | | | | |
|---|---|---|---|---|
| Deucravacitinib | 0.41 | 0.44 | 0.49 | 0.53 |
| 0.5M citric acid in | — | — | 25.00 | — |
| DEGEE | | | | |
| PEG 400 | 59.59 | 67.56 | 47.51 | 67.47 |
| DEGEE | 15.00 | 15.00 | — | 30.00 |
| TPGS | — | — | 15.00 | — |
| Propylene glycol | — | 15.00 | 10.00 | — |
| HPC | | 2.00 | 2.00 | 2.00 |
| PEG 3350 | 25.00 | — | — | — |

### Example 6—Preparation of Cream and Emulsified Gel Formulations

Cream formulations were prepared via the following method:
1. Solvents and preservative (which in these embodiments was benzyl alcohol) were added to a vessel and were stirred until homogenous.
2. If used, oil-based excipient(s), surfactant, and emulsion stabilizer were added to a separate vessel. Such components are provided in Table 6 and included Brij S2, Brij S721, cetyl alcohol, stearic acid, mineral oil, lanolin, glycerol, and medium chain triglyceride.
3. Deucravacitinib was added to the first vessel and the mixture was stirred until the deucravacitinib completely dissolved (based on visual observation).
4. Water or buffer was added to the first vessel dropwise while stirring. The oil phase in the second vessel was heated to 70° C. until melted.
5. The aqueous phase in the first vessel and homogenizer head were heated to 70° C.
6. The oil phase from the second vessel was added to the aqueous phase in the first vessel.
7. The two phases, now combined, were homogenized and stirred until cool.
8. pH adjustments were performed if needed.
9. Water (in an amount sufficient to make up the total target batch size) was added dropwise under stirring.
10. Stirring continued until the mixture was homogenous.

TABLE 6
Topical formulations of deucravacitinib - creams
(F10, F11, F12 and F13) and emulsified gel (F14)

| | F10 | F11 | F12 | F13 | F14 |
|---|---|---|---|---|---|
| Formulation | % w/w | | | | |
| Deucravacitinib | 0.31 | 0.50 | 0.46 | 0.69 | 0.55 |
| Water | 25.00 | — | 25.00 | — | — |
| Buffer (0.1N | — | 15.00 | — | 15.00 | 10.00 |
| hydrochloric acid in | | | | | |
| water) | | | | | |
| PEG 400 | 38.00 | 37.84 | 22.54 | 17.31 | 33.45 |
| DEGEE | — | 15.00 | 15.00 | 15.00 | 15.00 |
| Dimethyl Isosorbide | 15.00 | — | 15.00 | — | — |
| Propylene glycol | — | — | — | 20.00 | 10.00 |
| Brij S2 | 1.62 | 1.62 | 1.15 | 1.31 | — |
| Brij S721 | 2.94 | 2.94 | 2.85 | 2.69 | — |
| Cetyl alcohol | 5.00 | 5.00 | 4.00 | 5.50 | — |
| Stearic acid | — | — | 4.00 | — | — |
| Mineral oil | 10.13 | 10.10 | 8.00 | 4.50 | — |
| Glycerol | — | — | — | — | 5.00 |
| Medium chain | — | — | — | — | 17.00 |
| triglyceride | | | | | |
| Modified lanolin | — | — | — | 1.50 | — |
| Isopropyl myristate | — | — | — | 4.50 | — |
| Poloxamer 407 | — | — | — | — | 1.00 |
| Benzyl alcohol | 2.00 | 2.00 | 2.00 | 2.00 | 2.00 |
| Sodium hydroxide 0.5M | — | adjust | — | adjust | — |
| | | to pH | | to pH | |
| | | 4.5-6.0 | | 4.5-6.0 | |
| Ammonia solution 2.5% | — | — | — | — | adjust |
| | | | | | to pH |
| | | | | | 6.0-6.5 |
| Additional water | q.s. | q.s. | q.s. | q.s. | q.s. |

q.s.: quantity sufficient

### Example 7—Stability of Deucravacitinib Topical Formulations

Table 7 provides stability data for five formulations (F1, F6, F12, F13, and F14) when stored at 25° C. or 40° C., for 2 weeks or 4 weeks. Formulations F1, F6, F12, and F14 are described above. Purity of deucravacitinib remained above 99% for all of the formulations.

TABLE 7
Stability of deucravacitinib in topical formulations

Purity (%)

| Formulation | F1 | F6 | F12 | F13 | F14 |
|---|---|---|---|---|---|
| Initial | 99.84 | 99.83 | 99.82 | 99.73 | 99.87 |
| 25° C., 2 weeks | 99.79 | 99.86 | 99.83 | 99.77 | 99.88 |
| 40° C., 2 weeks | 99.84 | 99.83 | 99.80 | 99.64 | 99.84 |
| 25° C., 4 weeks | 99.78 | 99.80 | 99.74 | 99.70 | 99.85 |
| 40° C., 4 weeks | 99.78 | 99.85 | 99.79 | 99.64 | 99.46 |

### Example 8—In Vitro Skin Permeation and Penetration Testing

An experiment using flow-through diffusion cells was conducted to assess permeability and penetration of deucravacitinib across human skin excised at a thickness of 500±50 μm. Formulations were applied at 10 mg per square centimeter. Table 8 provides the amount of drug (mean±standard deviation) extracted from epidermal and dermal skin layers, along with the amount of drug measured in the receiver compartment, at 24 hours after application of the formulations.

Deucravacitinib concentrations (Drug Loading in Table 8) varied among the formulations, as each formulation was designed to carry appropriate drug load based on the solubilizing capacity of the solvent systems used. While deucravacitinib concentration was similar between F1 and F13 (0.66% w/w and 0.69% w/w, respectively), the amounts of deucravacitinib extracted from the skin layers, at 24 hours, were significantly higher for F13 when compared to F1. The amount of deucravacitinib in the receiver compartment was also higher for F13 when compared to F1. F12 led to the highest level of deucravacitinib in the receiver compartment, and the amount of deucravacitinib measured in epidermis and dermis were the second highest after F13. F14, with a deucravacitinib concentration of 0.55% w/w (which is slightly higher than the 0.50% w/w deucravacitinib in F12), led to lower amounts of deucravacitinib in the skin tissue and receiver compartment, compared to F12. Among these five formulations, F1 and F6 led to lower amounts of deucravacitinib in the skin layers and receiver compartment, compared to F12, F13, and F14.

A formulation that provides for a higher amount of drug recovered in the receiver compartment compared to the skin layers may be suitable for transdermal delivery. For a topical formulation of a drug that elicits effects by acting locally within the skin tissue, a low amount of drug in the receiver compartment (such as was observed with F1) may be desirable. In addition, a topical formulation that provides for delivery of an effective dose of the drug to the skin tissue (even if drug is also absorbed through the skin) can also be suitable for topical drug administration. For example, as shown for F12 and F14, the formulations can deliver a higher amount of deucravacitinib to the skin layers when compared to the amount of deucravacitinib recovered in the receiver compartment; such formulations may be useful for delivering an effective dose of deucravacitinib to the target skin tissue, with minimal side effects.

TABLE 8
Level of deucravacitinib in epidermis, dermis, and receiver,
measured 24 hours after application to excised skin (N = 5)

| | Drug Loading | Epidermis | Dermis | Receiver |
|---|---|---|---|---|
| | (deucravacitinib, | (deucravacitinib, | (deucravacitinib, | (deucravacitinib, |
| Formulation | % w/w) | ng) | ng) | ng) |
| F1 | 0.66 | 187 ± 16 | 225 ± 51 | 0.13 ± 0.03 |
| (aqueous gel) | | | | |
| F6 | 0.41 | 142 ± 58 | 320 ± 203 | 2.40 ± 4.68 |
| (ointment) | | | | |
| F12 | 0.50 | 1,779 ± 909 | 2,359 ± 858 | 56.21 ± 45.23 |
| (cream) | | | | |
| F13 | 0.69 | 4,912 ± 2,729 | 3,356 ± 1,728 | 39.69 ± 37.85 |
| (cream) | | | | |
| F14 | 0.55 | 934 ± 336 | 584 ± 345 | 11.84 ± 5.91 |
| (emulsified | | | | |
| gel) | | | | |

### Example 9—In Vivo Single-Dose Formulation Study in Minipigs

A single-dose study in minipigs was conducted. Each formulation was applied at a maximum feasible amount to achieve a thin uniform layer on designated dorsal surface areas of the animals, and the corresponding dose level was calculated. At each time point following administration (24 hours and 48 hours), skin samples were collected as punch biopsies of the treated area after tape stripping to remove stratum corneum. The biopsies were processed using heat to separate adipose tissue from epidermis and dermis layers of the skin. Table 9 provides the amounts of deucravacitinib measured within the skin at 24 hours and 48 hours after dermal administration, for five formulations.

F1 was applicable at the highest dose of 7.18 mg/kg, followed by F14 at 5.83 mg/kg. Following 24 hours after administration, F1 resulted in 195 ng/g of deucravacitinib in the skin, while F14 resulted in 170 ng/g of deucravacitinib in the skin. At 48 hours, these amounts increased to 264 ng/g and 416 ng/g for F1 and F14, respectively. F13 administered at 3.28 mg/kg resulted in 206 ng/g of deucravacitinib in the skin after 24 hours and 436 ng/g of deucravacitinib in the skin after 48 hours, and thus achieved drug levels comparable to F14. F12 exhibited the greatest variability compared to the other formulations. F6 provided the lowest amount of deucravacitinib in the skin after 48 hours.

TABLE 9
In vivo single-dose formulation study in minipig dermis (N = 3)

| | Dose of | Amount of deucravacitinib (ng) | |
|---|---|---|---|
| | deucravacitinib | per gram of skin tissue | |
| Formulation | (mg/kg) | 24 hours | 48 hours |
| F1 (aqueous gel) | 7.18 | 195 ± 15 | 264 ± 60 |
| F6 (ointment) | 4.50 | 178 ± 78 | 131 ± 70 |
| F12 (cream) | 4.47 | 177 ± 184 | 339 ± 244 |
| F13 (cream) | 3.28 | 206 ± 71 | 436 ± 7 |
| F14 (emulsified gel) | 5.83 | 170 ± 38 | 416 ± 20 |

### Example 10—In Vivo Multiple-Dose Formulation Study in Minipigs

Deucravacitinib formulations were topically administered to designated dorsal areas of minipigs twice daily, for 14 days. After the 14-day treatment, skin biopsies were taken from the treated area of the skin and were analyzed to determine the concentration of deucravacitinib in the treated skin. Table 10 provides the results for two formulations, F1 and F14.

TABLE 10
In vivo multiple-dose formulation study over a 14-day
treatment period in minipigs (N = 18; Male)

| | Dose Level | Amount of deucravacitinib |
|---|---|---|
| Formulation | (mg/kg/day) | (μg) per gram of skin tissue |
| F1 (aqueous gel) | 14.26 | 3.55 |
| F14 (emulsified gel) | 11.66 | 1.88 |

While this invention has been particularly shown and described with reference to preferred embodiments thereof, it will be understood by those skilled in the art in light of the present disclosure that various changes in form and detail may be made therein without departing from the scope of the invention encompassed by the appended claims.

What is claimed is:

1. A topical pharmaceutical composition comprising deucravacitinib and two ether solvents.

2. The topical pharmaceutical composition according to claim 1, wherein the two ether solvents are selected from polyethylene glycol 400, diethylene glycol monoethyl ether, dimethyl isosorbide, d-α-tocopheryl polyethylene glycol 1000 succinate, and propylene glycol.

3. The topical pharmaceutical composition according to claim 1, wherein the two ether solvents comprise polyethylene glycol 400.

4. The topical pharmaceutical composition according to claim 1, wherein the two ether solvents are polyethylene glycol 400 and diethylene glycol monoethyl ether.

5. The topical pharmaceutical composition according to claim 1, wherein the two ether solvents are polyethylene glycol 400 and dimethyl isosorbide.

6. The topical pharmaceutical composition according to claim 1, wherein the two ether solvents are polyethylene glycol 400 and dimethyl isosorbide, and wherein the topical pharmaceutical composition further comprises diethylene glycol monoethyl ether.

7. A topical pharmaceutical composition comprising deucravacitinib and three ether solvents.

8. The topical pharmaceutical composition according to claim 7, wherein the three ether solvents are selected from polyethylene glycol 400, diethylene glycol monoethyl ether, dimethyl isosorbide, d-α-tocopheryl polyethylene glycol 1000 succinate, and propylene glycol.

9. The topical pharmaceutical composition according to any one of claims 1-8, further comprising a thickening agent.

10. The topical pharmaceutical composition according to claim 9, wherein the thickening agent is a carbomer.

11. The topical pharmaceutical composition according to claim 9, wherein the thickening agent is a carbomer derivative.

12. The topical pharmaceutical composition according to any one of claims 1-11, further comprising an acidic buffer.

13. A topical pharmaceutical composition comprising deucravacitinib, an ether solvent, and an acidic buffer.

14. The topical pharmaceutical composition according to claim 13, wherein the ether solvent is selected from polyethylene glycol 400, diethylene glycol monoethyl ether, dimethyl isosorbide, d-α-tocopheryl polyethylene glycol 1000 succinate, and propylene glycol.

15. The topical pharmaceutical composition according to claim 13, wherein the ether solvent is polyethylene glycol 400.

16. The topical pharmaceutical composition according to claim 13, wherein the ether solvent is diethylene glycol monoethyl ether.

17. The topical pharmaceutical composition according to claim 13, wherein the ether solvent is dimethyl isosorbide.

18. The topical pharmaceutical composition according to any one of claims 16 and 17, wherein the topical pharmaceutical composition further comprises polyethylene glycol 400.

19. The topical pharmaceutical composition according to any one of claims 1-11, wherein the topical pharmaceutical composition is a non-aqueous gel.

20. The topical pharmaceutical composition according to any one of claims 1-11, wherein the topical pharmaceutical composition is an ointment.

21. The topical pharmaceutical composition according to any one of claims 9-18, wherein the topical pharmaceutical composition is an aqueous gel.

22. The topical pharmaceutical composition according to any one of claims 12-18, wherein the topical pharmaceutical composition is a cream.

23. The topical pharmaceutical composition according to any one of claims 1-22, wherein the deucravacitinib is present in an amount of: from about 0.075% w/w to about 1.1% w/w, based on total weight of the topical pharmaceutical composition.

24. A method of treating psoriasis in a subject, the method comprising administering to the subject the topical pharmaceutical composition according to any one of claims 1-23.

25. A method of treating psoriatic arthritis in a subject, the method comprising administering to the subject the topical pharmaceutical composition according to any one of claims 1-23.

26. A method of treating systemic lupus erythematosus in a subject, the method comprising administering to the subject the topical pharmaceutical composition according to any one of claims 1-23.

27. A method of treating alopecia areata in a subject, the method comprising administering to the subject the topical pharmaceutical composition according to any one of claims 1-23.
